(19) United States
(12) Patent Application Publication
(10) Pub. No.: US 2011/0313153 A1
(43) Pub. Date: Dec. 22, 2011

(54) 5-HT4 INHIBITORS FOR TREATING AIRWAY DISEASES, IN PARTICULAR ASTHMA

(75) Inventors: Ann Meulemans, Turnhout (BE); Leen Thielemans, Turnhout (BE); Joris De Maeyer, Turnhout (BE); Jan Schuurkes, Turnhout (BE)

(73) Assignee: SHIRE-MOVETIS N.V.

(21) Appl. No.: 13/146,962

(22) PCT Filed: Jan. 28, 2010
(86) PCT No.: PCT/EP10/51028

§ 371 Date: Jul. 29, 2011

(30) Foreign Application Priority Data
Jan. 30, 2009 (GB) 0901487.9

(51) Int. Cl. C07D 498/04; C07D 451/02; C07D 471/04; C07D 405/12; C07D 401/12

(52) U.S. Cl. 544/89; 546/197; 546/201; 546/121; 546/126; 546/199

(57) ABSTRACT

The invention relates generally to the treatment of diseases of the respiratory system such as asthma and chronic obstructive pulmonary disease. More particularly, the present invention relates to methods of treating and preventing asthmatic airway inflammation. The treatment involves the administration of a 5-HT4 receptor antagonist to the subject in need thereof; more in particular the administration of aroylated 4-aminomethylpiperidine derivatives as defined herein. Other aspects of the invention are directed to compositions for treating or preventing respiratory disorders, including pharmaceutical compositions.

### FIELD OF THE INVENTION

The invention relates generally to the treatment of diseases of the respiratory system such as asthma and chronic obstructive pulmonary disease. More particularly, the present invention relates to methods of treating and preventing asthmatic airway inflammation. The treatment involves the administration of a 5-HT4 receptor antagonist to the subject in need thereof; more in particular the administration of aroylated 4-aminomethylpiperidines as defined herein below.

Other aspects of the invention are directed to compositions for treating or preventing respiratory disorders, including pharmaceutical compositions.

### BACKGROUND TO THE INVENTION

Damage or infection to the lungs can give rise to a wide range of diseases of the respiratory system (respiratory disorders or airway diseases). A number of these diseases are of great public health importance. Airway diseases include Acute Lung Injury, Acute Respiratory Distress Syndrome (ARDS), occupational lung disease, lung cancer, tuberculosis, fibrosis, pneumoconiosis, pneumonia, emphysema, Chronic Bronchitis, Chronic Obstructive Pulmonary Disease (COPD) and asthma.

Among the most common airway diseases is asthma. Asthma is generally defined as an inflammatory disorder of the airways with clinical symptoms arising from intermittent airflow obstruction. It is characterized clinically by paroxysms of wheezing, dyspnea and cough. It is a chronic disabling disorder that appears to be increasing in prevalence and severity. It is estimated that 15% of children and 5% of adults in the population of developed countries suffer from asthma. Therapy should therefore be aimed at controlling symptoms so that normal life is possible and at the same time provide basis for treating the underlying inflammation.

COPD is a term that refers to a large group of lung diseases that can interfere with normal breathing. Current clinical guidelines define COPD as a disease state characterized by airflow limitation that is not fully reversible. The airflow limitation is usually both progressive and associated with an abnormal inflammatory response of the lungs to noxious particles and gases. The most important contributory source of such particles and gases, at least in the western world, is tobacco smoke. COPD patients have a variety of symptoms, including cough, shortness of breath, and excessive production of sputum; such symptoms arise from dysfunction of a number of cellular compartments, including neutrophils, macrophages, and epithelial cells. The two most important conditions covered by COPD are chronic bronchitis and emphysema.

Chronic bronchitis is a long-standing inflammation of the bronchi which causes increased production of mucous and other changes. The patients' symptoms are cough and expectoration of sputum. Chronic bronchitis can lead to more frequent and severe respiratory infections, narrowing and plugging of the bronchi, difficult breathing and disability.

Emphysema is a chronic lung disease which affects the alveoli and/or the ends of the smallest bronchi. The lung loses its elasticity and therefore these areas of the lungs become enlarged. These enlarged areas trap stale air and do not effectively exchange it with fresh air. This results in difficult breathing and may result in insufficient oxygen being delivered to the blood. The predominant symptom in patients with emphysema is shortness of breath.

The present invention relates to the application of a selective 5-HT4 receptor antagonist in the treatment of airway diseases, and is the first demonstration in the perifery of an effect of a 5-HT4 receptor antagonist per se, i.e. without prior activation of 5-HT4 receptors with exogenously applied agonists.

In the periphery, the 5-HT4 receptor (5-HT4 R) has mainly been studied in the gastrointestinal (GI) tract. Activation of these GI 5-HT4 receptors results in GI prokinetic effects. Consistent with this activity, 5-HT4 R agonists have been and are being developed to treat GI hypomotility disorders (Sanger et al., 2008; Development of drugs for gastrointestinal motor disorders: translating science to clinical need. Neurogastroenterol Motil, 20 (3), 177-84.). Despite the clear effects of 5-HT4 R agonists in the GI tract, an effect of a 5-HT4 R antagonist per se has, to the best of our knowledge, never been observed, nor in healthy animal models nor in disease models. Thus far, 5-HT4 R antagonists have only proven to be capable to suppress or inverse the 5-HT4 R-mediated prokinetic effects of serotonin or 5-HT4 R agonists in the GI tract.

For example piboserod (SB 207266), an indazole amide 5-HT4 R antagonist, antagonizes the 5-HT4 R-mediated effects of serotonin (5-HT) in the GI tract (Sanger et al., 2000; “Increased defecation during stress or after 5-hydroxytryptophan: selective inhibition by the 5-HT(4) receptor antagonist, SB-207266.” Br J Pharmacol; 130(3):706-12; and Bharucha et al., 2000; “Effects of a serotonin 5-HT(4) receptor antagonist SB-207266 on gastrointestinal motor and sensory function in humans.”Gut, 47(5):667-74), but it does not seem to affect normal bowel motility in animals or humans (Sanger et al., 1998; “SB-207266: 5-HT4 receptor antagonism in human isolated gut and prevention of 5-HT-evoked sensitization of peristalsis and increased defaecation in animal models.” Neurogastroenterol Motil, 10(4):271-9).

Also the aroylated 4-aminomethylpiperidines 5-HT4 receptor antagonists (hereinafter also referred to as the compounds) of the present invention (e.g. compound M0014) were capable to suppress or inverse the 5-HT4 R-mediated prokinetic activity of serotonin or 5-HT4 R agonists in the GI tract (data not shown). For example, in conscious dogs, low doses of M0014 reversed the selective serotonin re-uptake inhibitor (SSRI)-induced loss of fundic compliance. Also in a dog model of delayed gastric emptying of a liquid meal, M0014 potently inhibited the 5-HT4 R agonist-induced acceleration of gastric emptying. As a final example, the compound reversed the 5-HT4 R agonist-induced stimulation of canine antral motility, measured with chronically implanted strain gauges.

Similar to SB-207266, the 5-HT4 R antagonist M0014 had by itself no effect on the studied GI functions mentioned above. Taken together, no effects other than inhibition of 5-HT-induced effects were observed in the GI tract.

Also in airway diseases like asthma, up till now, no direct effects have been observed for 5-HT4 R antagonists, per se. In a first series of publications, the effect of stimulating the 5-HT4 R on asthmatic inflammatory responses could be established in human airway epithelial cells (Bayer et al., 2007; “Serotoninergic receptors on human airway epithelial cells.” Am J Respir Cell Mol Biol. 36(1):85-93), dendritic cells (Idzko et al., 2004;“The serotoninergic receptors of human dendritic cells: identification and coupling to cytokine release.” J Immunol. 172(10):6011-9.) and monocytes (Durk et al., 2005; “5-Hydroxytryptamine modulates cytokine and chemokine production in LPS-primed human monocytes via stimulation of different 5-HTR subtypes.” Int Immunol. 17(5):599-606). In those studies where 5-HT4 R antagonists, such as RS 39604, were used, the 5-HT4 R antagonists was only shown to inhibit the effects of 5-HT but again and similar to the GI observations, no effect of the 5-HT4 R antagonist per se was described.

Contrary to the beneficial effects of the 5-HT4 R antagonists presented in the present application, 5-HT4 R agonists for use in the treatment of disorders involving bronchocontraction were extensively described, such as for example in the PCT publications WO 00/76500 and WO 02/36113. Again, in studies on the involvement of the 5-HT4 R in the bronchocontractile effects of serotonin (Dupont et al., 1999; “The effects of 5-HT on cholinergic contraction in human airways in vitro.” Eur Respir J 14: 642-649) the 5-HT4 R antagonist GR125487D could only antagonize the 5-HT-induced facilitation of cholinergic contractions that was mimicked by the 5-HT4 R agonist RS 67333, but again no effect of the antagonist per se was described. In the latter paper, high concentrations of 5-HT were needed (10 μM to 0.3 mM) in order to see an effect and a high concentration of GR 125487D (1 μM) was used to antagonize this effect. The involvement of the 5-HT4 R in these effects therefore needs confirmation.

Only compounds which combine antagonism of both muscarinic receptors and serotonin receptors, such as for example described in PCT publication WO01/64631 have thus far been found effective in reducing serotonin-induced bronchocontraction and accordingly useful in the treatment of disorders involving bronchocontraction such as asthma. Such compounds have no selectivity for either the muscarinic or the serotonin receptors alone, but address both the 5-HT4 receptors and the muscarinic receptors to reduce serotonin induced airway smooth muscle contraction. An effect on the contractile response by a selective 5-HT4 R antagonist alone (without additional antagonism of muscarinic receptors) and per se (without pre-contraction with an agonist), and as presented in the present application, was thus not shown.

Unexpectedly and in contrast to the lack of effect that was described for 5-HT4 R antagonists in the GI tract and in inflammatory and mechanistic in vitro studies for asthma, we now clearly show an effect of the compounds per se: i.e. they inhibit inflammatory cell recruitment in in vivo mouse models of asthma and lung inflammation, and they inhibit cytokine production and improve respiratory function in an in vivo mouse model of asthma.

### BRIEF DESCRIPTION OF THE DRAWINGS

FIG. 1. Local administration of M0014 suppresses asthma features. Mice were sensitized by i.p. injection of OVA/alum on days 0 and 7 and were exposed on days 19-21 to OVA aerosols. Prior to each aerosol, mice received an i.t. injection of vehicle or M0014 at 0.1, 0.4 or 4 nM. Legend labels (e.g. OVA/M0014/OVA) indicate sensitization/treatment/challenge. BAL fluid was analyzed by flow cytometry (A). Cytokine production in BAL fluid (B) and in MLN cells re-stimulated in vitro for 4 days with OVA (C-D). Data are mean±SEM; n=8 mice per group.

FIG. 2. BHR (Bronchial Hyperreactivity) to various doses of i.v. metacholine was assessed for changes in dynamic resistance (top) and lung compliance (bottom) and BHR to inhaled metacholine for PenH responses was assessed 24 hours after the last antigen exposure were measured.

FIG. 3. The effect of M0014 on total cell recruitment (left upper panel), mononuclear cell number (right upper panel) and neutrophil recruitment (bottom left panel) in BALBc mice. Each column represents mean+standard error of the mean from 3-6 animals. There was a significant effect of M0014 on total and neutrophil cell number (<0.05, cf zymosan alone).

### DETAILED DESCRIPTION OF THE INVENTION

This invention relates to methods and compositions for treating and preventing diseases of the respiratory system, and is based on the finding that selective 5-HT4 R antagonists, such as the benzoate derivatives; the indole amides; the indole esters and the imidazopyridine, indazole, and benzimidazole derivatives described in (Langlois et al., 2003; “5-HT4 Receptor ligands: Applicatons and new prospects.” J. Med. Chem., 46(3):319-344), bring about a considerable improvement with regard to the respiratory function in chronic airway disorders like asthma and COPD.

It is accordingly a first aspect of the present invention to provide a selective 5-HT4 R antagonist for use in the treatment and/or prevention of airway diseases; in particular for use in the treatment and/or prevention of asthmatic airway inflammation.

In particular embodiment the 5-HT4 R antagonist for use in the treatment and/or prevention of airway diseases is selected from the group consisting of;

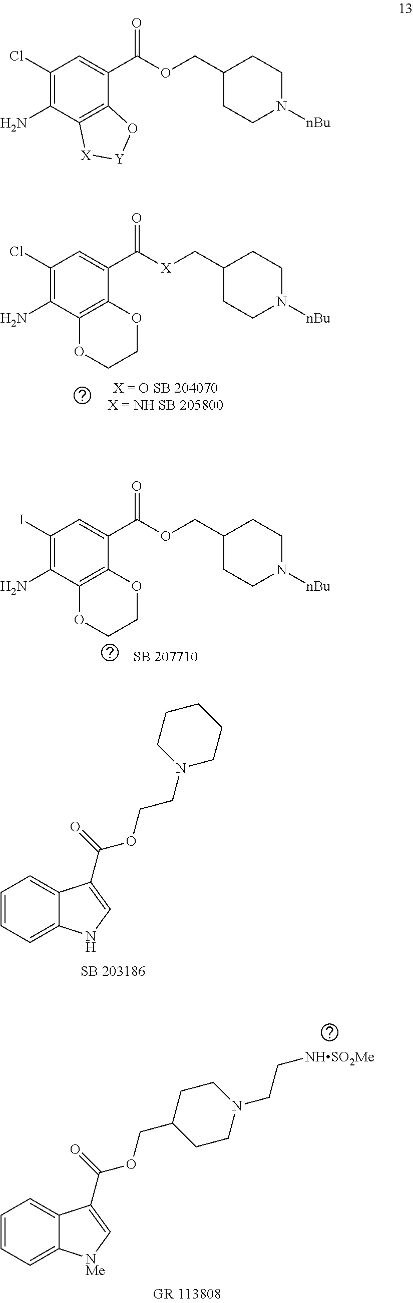

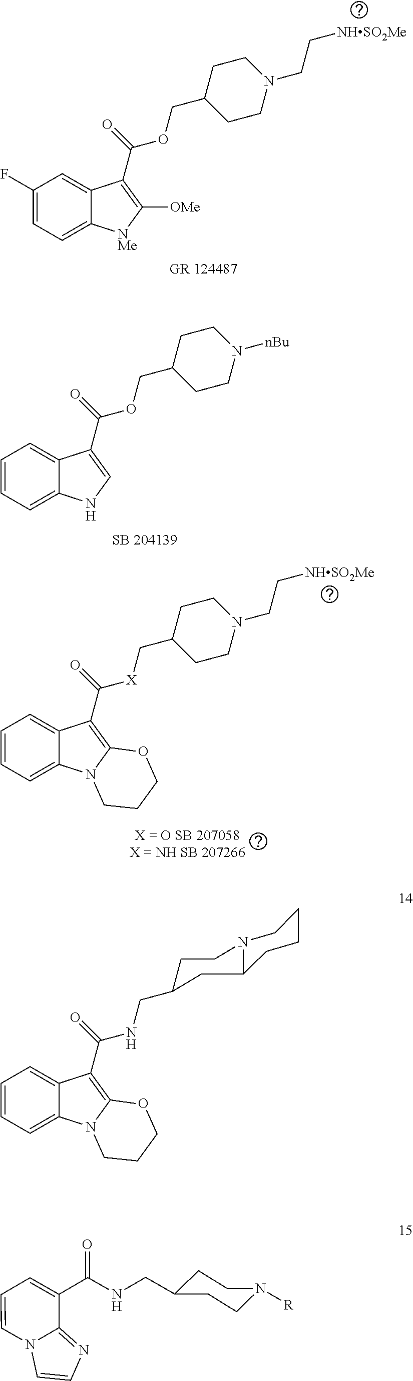

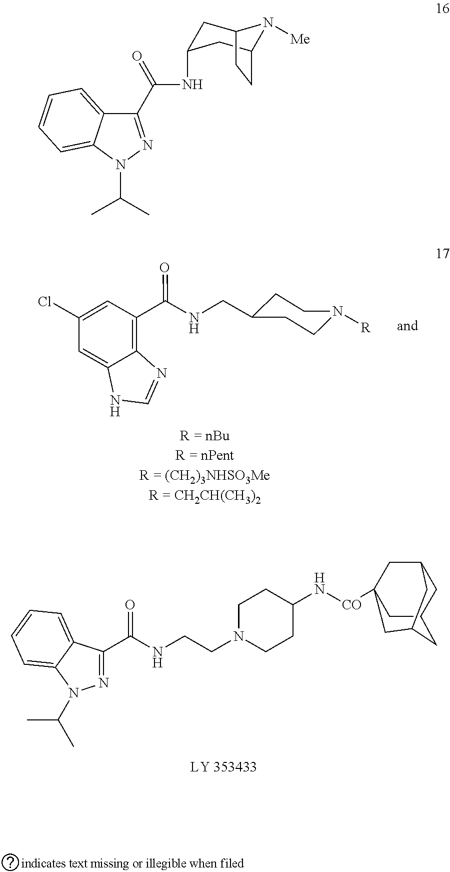

In a further embodiment, the 5-HT4 R antagonsist for use in the treatment and/or prevention of airway diseases is selected from the class of aroylated 4-aminomethylpiperidines as described in the PCT patent publications WO2005003121; WO2005003122; WO2005003124, WO2005000837 & WO2005000838; and generally represented as the compounds of formula (I)

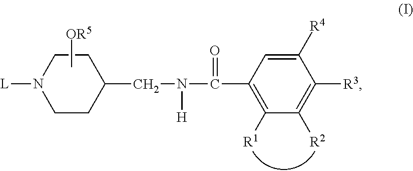

a stereochemically isomeric form thereof, an N-oxide form thereof, or a pharmaceutically acceptable acid or base addition salt thereof, wherein —R¹-R²-is a bivalent radical of formula

—O—CH₂—O— (a-1),

—O—CH2-CH2- (a-2),

—O—CH2-CH2-O— (a-3),

—O—CH2-CH2-CH2- (a-4),

—O—CH2-CH2-CH2-O— (a-5),

—O—CH2-CH2-CH2-CH2- (a-6),

—O—CH2-CH2-CH2-CH2-O— (a-7),

—O—CH2-CH2-CH2-CH2-CH2- (a-8),

wherein in said bivalent radicals optionally one or two hydrogen atoms on the same or a different carbon atom may be replaced by C₁₋₆alkyl or hydroxy,
R³ is hydrogen, halo, C₁₋₆alkyl or C₁₋₆alkyloxy;
R⁴ is hydrogen, halo, C₁₋₆alkyl; C₁₋₆alkyl substituted with cyano, or C₁₋₆alkyloxy; C₁₋₆alkyloxy; cyano; amino or mono or di(C₁₋₆alkyl)amino;
R⁵ is hydrogen or C₁₋₆alkyl and the —OR⁵ radical is situated at the 3- or 4-position of the piperidine moiety; L is hydrogen, or L is a radical of formula

-Alk-R⁶ (b-1),

-Alk-X—R⁷ (b-2),

-Alk-Y—C(═O)—R⁹ (b-3),

-Alk-Z—C(═O)—NR¹¹R¹² (b-4)

-Alk-C(═O)—NH—C(═O)—R¹³ (b-5),

-Alk-C(═O)—NH—SO₂—R¹³ (b-6),

-Alk-SO₂—NH—C(═O)—R¹³ (b-7),

-Alk-SO₂—NH—SO₂—R¹³ (b-8),

wherein each Alk is C₁₋₁₂alkanediyl; and
R⁶ is hydrogen; hydroxy; cyano; C₃₋₆cycloalkyl; C₁₋₆alkylsulfonylamino; aryl; aminosulfonyl optionally substituted with C₁₋₄alkyl, C₃₋₆cycloalkyl or phenyl; or Het;
R⁷ is C₁₋₆alkyl; C₁₋₆alkylsulfonyl; C₁₋₆alkyl substituted with hydroxy; C₃₋₆cycloalkyl; aryl or Het;
R⁹ is hydrogen, C₁₋₆alkyl, C₁₋₆alkylsulfonylamino, C₃₋₆cycloalkyl, hydroxy or aryl;
X is O, S, SO₂ or NR⁸; said R⁸ being hydrogen or C₁₋₆alkyl; R⁹ is hydrogen, C₁₋₆alkyl, C₁₋₆alkylsulfonylamino, C₃₋₆cycloalkyl, hydroxy or aryl;
Y is a direct bond, O, S, or NR¹⁰ wherein R¹⁰ is hydrogen or C₁₋₆alkyl;
Z is a direct bond, O, S, or NR¹⁰ wherein R¹⁰ is hydrogen or C₁₋₆alkyl;
R¹¹ and R¹² each independently are hydrogen, C₁₋₆alkyl, C₃₋₆cycloalkyl, or R¹¹ and R¹² combined with the nitrogen atom bearing R¹¹ and R¹² may form a pyrrolidinyl, piperidinyl, piperazinyl or 4-morpholinyl ring both being optionally substituted with C₁₋₆alkyl;
R¹³ is C₁₋₆alkyl or phenyl;
aryl represents unsubstituted phenyl or phenyl substituted with 1, 2 or 3 substituents each independently selected from halo, hydroxy, C₁₋₆alkyl, C₁₋₆alkyloxy, C₁₋₆alkylcarbonyl, nitro, trifluoromethyl, amino, aminocarbonyl, hydroxycarbonyl, and aminosulfonyl; and
Het is furanyl; furanyl substituted with C₁₋₆alkyl or halo; tetrahydrofuranyl; tetrahydrofuranyl substituted with C₁₋₆alkyl; dioxolanyl; dioxolanyl substituted with C₁₋₆alkyl; dioxanyl; dioxanyl substituted with C₁₋₆alkyl; tetrahydropyranyl; tetrahydropyranyl substituted with C₁₋₆alkyl; 2,3-dihydro-2-oxo-1H-imidazolyl; 2,3-dihydro-2-oxo-1H-imidazolyl substituted with one or two substituents each independently selected from halo, or C₁₋₆alkyl; pyrrolidinyl; pyrrolidinyl substituted with one or two substituents each independently selected from halo, hydroxy, or C₁₋₆alkyl; pyridinyl; pyridinyl substituted with one or two substituents each independently selected from halo, hydroxy, C₁₋₆alkyl; pyrimidinyl; pyrimidinyl substituted with one or two substituents each independently selected from halo, hydroxy, or C₁₋₆alkyl; pyridazinyl; pyridazinyl substituted with one or two substituents each independently selected from hydroxy, C₁₋₆alkyloxy,
C₁₋₆alkyl or halo; pyrazinyl; pyrazinyl substituted with one ore two substituents each independently selected from hydroxy, C₁₋₆alkyloxy, C₁₋₆alkyl or halo.; morpholinyl; morpholinyl substituted with C₁₋₆alkyl; tetrazolyl; tetrazolyl substituted with halo, hydroxy, or C₁₋₆alkyl; pyrazolyl; pyrazolyl substituted with halo, hydroxy, or C₁₋₆alkyl; isoxazolyl; isoxazolyl substituted with halo, hydroxy, or C₁₋₆alkyl; isothiazolyl; isothiazolyl substituted with halo, hydroxy, or C₁₋₆alkyl; 2,4-dioxo-imidazolidinyl; 2,4-dioxo-imidazolidinyl substituted with one or two substituents each independently selected from halo, or C₁₋₆alkyl; oxazolyl; oxazolyl substituted with halo, hydroxy, or C₁₋₆alkyl; thiazolyl; thiazolyl substituted with halo, hydroxy, or C₁₋₆alkyl; or pyranyl; pyranyl substituted with halo, hydroxy, or C₁₋₆alkyl.

In one embodiment of the present invention, the compounds for use in the treatment of the airway diseases are selected from those compounds of formula (I), wherein one or more of the following restrictions apply:
R³ is hydrogen, halo, or C₁₋₆alkyl;
R⁴ is C₁₋₆alkyl; C₁₋₆alkyl substituted with cyano, or C₁₋₆alkyloxy; C₁₋₆alkyloxy; cyano; amino or mono or di(C₁₋₆alkyl)amino;
L is hydrogen, or L is a radical of formula

-Alk-R⁶ (b-1),

-Alk-X—R⁷ (b-2),

-Alk-Y'C(═O)—R⁹ (b-3), or

-Alk-Z—C(═O)—NR¹¹R¹² (b-4)

wherein each Alk is C₁₋₁₂alkanediyl; and
R⁶ is hydrogen; hydroxy; cyano; C₃₋₆cycloalkyl; C₁₋₆alkylsulfonylamino; aryl; or Het;
R⁷ is C₁₋₆alkyl; C₁₋₆alkyl substituted with hydroxy; C₃₋₆cycloalkyl; aryl or Het;
R⁹ is hydrogen, C₁₋₆alkyl, C₃₋₆cycloalkyl, hydroxy or aryl;
Y is a direct bond, or NR¹⁰ wherein R¹⁰ is hydrogen or C₁₋₆alkyl;
Z is a direct bond, O, S, or NR¹⁰ wherein R¹⁰ is hydrogen or C₁₋₆alkyl;
R¹¹ and R¹² each independently are hydrogen, C₁₋₆alkyl, C₃₋₆cycloalkyl, or R¹¹ and R¹² combined with the nitrogen atom bearing R¹¹ and R¹² may form a pyrrolidinyl, piperidinyl, piperazinyl or 4-morpholinyl ring both being optionally substituted with C₁₋₆alkyl;
aryl represents unsubstituted phenyl or phenyl substituted with 1, 2 or 3 substituents each independently selected from halo, hydroxy, C₁₋₆alkyl, C₁₋₆alkyloxy, C₁₋₆alkylcarbonyl, nitro, trifluoromethyl, amino, aminocarbonyl, and aminosulfonyl; and
Het is furanyl; furanyl substituted with C₁₋₆alkyl or halo; tetrahydrofuranyl; tetrahydrofuranyl substituted with C₁₋₆alkyl; dioxolanyl; dioxolanyl substituted with C₁₋₆alkyl; dioxanyl; dioxanyl substituted with C₁₋₆alkyl; tetrahydropyranyl; tetrahydropyranyl substituted with C₁₋₆alkyl; 2,3-dihydro-2-oxo-1H-imidazolyl; 2,3-dihydro-2-oxo-1H-imidazolyl substituted with one or two substituents each independently selected from halo, or C₁₋₆alkyl; pyrrolidinyl; pyrrolidinyl substituted with one or two substituents each independently selected from halo, hydroxy, or C₁₋₆alkyl; pyridinyl; pyridinyl substituted with one or two substituents each independently selected from halo, hydroxy, C₁₋₆alkyl; pyrimidinyl; pyrimidinyl substituted with one or two substituents each independently selected from halo, hydroxy, or C₁₋₆alkyl; pyridazinyl; pyridazinyl substituted with one or two substituents each independently selected from hydroxy, C₁₋₆alkyloxy,
C₁₋₆alkyl or halo; pyrazinyl; pyrazinyl substituted with one ore two substituents each independently selected from hydroxy, C₁₋₆alkyloxy, C₁₋₆alkyl or halo.

An interesting group of compounds for use in the treatment of the airway diseases are selected from those compounds of formula (I), wherein one or more of the following restrictions apply:
the —OR⁵ radical is situated at the 3- or 4-position of the piperidine moiety;
the absolute configuration of the piperidine moiety is (3S,4S);
L is a radical of formula (b-1), (b-2), (b-6) or (b-8); more in particular L is a radical of formula (b-2);
Alk is C₁₋₄alkanediyl; 1,3-propanediyl or 1,4-butanediyl; more in particular Alk is C₁₋₄alkanediyl;
—R¹-R²-is a bivalent radical of formula (a-5);
R³ is hydrogen, halo, or C₁₋₄alkyl; more in particular R³ is hydrogen;
R⁴ is halo or C₁₋₆alkyl; more in particular R⁴ is C₁₋₆alkyl
R⁵ is hydrogen or C₁₋₆alkyl; more in particular R⁵ is hydrogen and the —OR⁵ radical is situated at the 3-position of the piperidine moiety having the trans configuration;
R⁶ is Het, aminosulfonyl, or aminosulfonyl substituted with C₁₋₄alkyl or phenyl; more in particular R⁶ is Het;
R⁷ is aryl or C₁₋₆alkyl;
R¹³ is C₁₋₄alkyl;
Het is morpholinyl; pyrazolyl substituted with hydroxy;
isoxazolyl substituted with hydroxy; 2,4-dioxo-imidazolidinyl; tetrazolyl; or tetrazolyl substituted with hydroxy

In a more particular embodiment the aroylated 4-aminomethylpiperidine derivatives used according to the invention consists of the compound of formula (I) wherein;
—R¹-R²— is a radical of formula (a-5);
R³ is hydrogen;
R⁴ is methyl;
R⁵ is hydrogen;
L is a radical of formula (b-2), wherein X is O, Alk is C₁₋₄alkanediyl and R⁷ is C₁₋₆alkyl; and,

including the stereo-isomeric forms, solvates and pharmaceutically acceptable addition salts thereof.

In an even further embodiment the benzofuran carboxamide derivative used according to the invention consists of

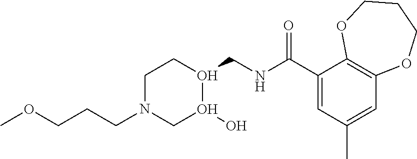

(3S-trans)-8-methyl-3,4-dihydro-3H-benzo[b][1,4]dioxepine-6-carboxylic acid [3-hydroxy-1-(3-methoxy-propyl)-piperidine-4-ylmethyl]-amide, in the experimental part hereinafter also referred to as compound M0014, including the stereo-isomeric forms, solvates and pharmaceutically acceptable addition salts thereof.

As is evident from the pharmacological examples in the PCT patent publications WO2005003121; WO2005003122; WO2005003124, WO2005000837 & WO2005000838; the 5-HT4 receptor antagonist as provided herein are selective 5-HT4 receptor antagonists based on a HEK293-5-HT4 binding assay.

In a further embodiment the present invention provides the use an 5-HT4 receptor antagonist such as the aroylated 4-aminomethylpiperidine derivatives as defined hereinbefore, in the manufacture of a medicament for the treatment and/or prevention of an airway disease; in particular for the treatment and/or prevention of chronic airway disorders like asthma and CPOD; more in particular in the treatment of asthmatic airway inflammation. In a particular embodiment, the present invention provides the use of a benzofuran carboxamide derivative as defined hereinbefore, in the manufacture of a medicament for the treatment and/or prevention of an airway disease; in particular for the treatment and/or prevention of chronic airway disorders like asthma and CPOD; more in particular in the treatment of asthmatic airway inflammation. In a further embodiment, the present invention provides the use of (3S-trans)-8-methyl-3,4-dihydro-3H-benzo[b][1,4]dioxepine-6-carboxylic acid [3-hydroxy-1-(3-methoxy-propyl)-piperidine-4-ylmethyl]-amide (also known as M0014), in the manufacture of a medicament for the treatment and/or prevention of an airway disease; in particular for the treatment and/or prevention of chronic airway disorders like asthma and CPOD; more in particular in the treatment of asthmatic airway inflammation.
As used herein with respect to a substituting radical, and unless otherwise stated, the term “alkyl” relates to a fully saturated hydrocarbon, including straight and branched chains, wherein for example a C₁₋₄alkyl represents a straight or branched fully saturated hydrocarbon radicals having from 1 to 4 carbon atoms such as for example, methyl, propyl, 1-methyl-ethyl and the like.
As used herein with respect to a substituting radical, and unless otherwise stated, the term “alkanediyl” relates to a bivalent straight or branched saturated hydrocarbon wherein for example a C₁₋₁₂alkanediyl represents bivalent straight or branched chain hydrocarbon radicals containing from 1 to 12 carbon atoms such as, for example, methanediyl, 1,2-ethanediyl, 1,3-propanediyl, 1,4-butanediyl, 1,5-pentanediyl, 1,6-hexanediyl, 1,7-heptanediyl, 1,8-octanediyl, 1,9-nonanediyl, 1,10-decanediyl, 1,11-undecanediyl, 1,12-dodecanediyl and the branched isomers thereof.
As used herein with respect to a substituting radical, and unless otherwise stated, the term “halogen” refers to any atom selected from the group consisting of fluorine, chlorine, bromine and iodine.

In view of the utility of the compounds according to the invention, there is provided a method for the treatment of an animal, for example, a mammal including humans, suffering from an airway disease, which comprises administering an effective amount of a compound according to the present invention, i.e. a 5-HT4 receptor antagonist, to said animal.

Said method comprising the systemic or topical administration of an effective amount of a compound according to the invention, to animals, including humans.

The compounds according to the invention can be prepared and formulated into pharmaceutical compositions by methods known in the art and in particular according to the methods described in the published patent specifications WO2005003121; WO2005003122; WO2005003124, WO2005000837 & WO2005000838 mentioned herein and incorporated by reference.

To prepare the aforementioned pharmaceutical compositions, a therapeutically effective amount of the particular compound, optionally in addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous, or parenteral administration; or topical administration such as via inhalation, a nose spray, eye drops or via a cream, gel, shampoo or the like.

The pharmaceutical compositions of the present invention can be prepared by any known or otherwise effective method for formulating or manufacturing the selected product form. Methods for preparing the pharmaceutical compositions according to the present invention can be found in “Remington's Pharmaceutical Sciences”, Mid. Publishing Co., Easton, Pa., USA.

For example, the compounds can be formulated along with common excipients, diluents, or carriers, and formed into oral tablets, capsules, sprays, mouth washes, lozenges, treated substrates (e.g., oral or topical swabs, pads, or disposable, non-digestible substrate treated with the compositions of the present invention); oral liquids (e. g. suspensions, solutions, emulsions), powders, or any other suitable dosage form.

Non-limiting examples of suitable excipients, diluents, and carriers can be found in “Handbook of Pharmaceutical Excipients”, Second edition, American Pharmaceutical Association, 1994 and include: fillers and extenders such as starch, sugars, mannitol, and silicic derivatives; binding agents such as carboxymethyl cellulose and other cellulose derivatives, alginates, gelatin, and polyvinyl pyrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as acetyl alcohol, glycerol monostearate; adsorptive carriers such as kaolin and bentonite; carriers such as propylene glycol and ethyl alcohol, and lubricants such as talc, calcium and magnesium stearate, and solid polyethyl glycols.

As another aspect of the present invention a combination of a 5-HT4 R antagonist, such as the benzofuran carboxamide derivative as defined hereinbefore, with another agent used in the treatment of chronic airway disorders like asthma and COPD is envisaged.

For the treatment of chronic airway disorders like asthma and CPOD, in particular for the treatment and/or prevention of asthmatic airway inflammation; the compounds of the present invention may advantageously be employed in combination with other agents used in the treatment of asthma. Examples of other agents used in the treatment of asthma include long-term control medications, quick-relief (rescue) medications and medications to treat allergies.

Long-Term Control Medications
Inhaled corticosteroids such as fluticasone (Flovent Diskus), budesonide (Pulmicort), triamcinolone (Azmacort), flunisolide (Aerobid), beclomethasone (Qvar) and others. These medications reduce airway inflammation and are the most commonly used long-term asthma medication.
Long-acting beta-2 agonists (LABAs) such as salmeterol (Serevent Diskus) and formoterol (Foradil Aerolizer). These inhaled medications, called long-acting bronchodilators, open the airways and reduce inflammation. They are often used to treat persistent asthma in combination with inhaled corticosteroids.
Leukotriene modifiers such as montelukast (Singulair), zafirlukast (Accolate) and zileuton (Zyflo CR). These inhaled medications work by opening airways, reducing inflammation and decreasing mucus production.
Cromolyn and nedocromil (Tilade). These inhaled medications reduce asthma signs and symptoms by decreasing allergic reactions.
Theophylline, a daily pill that opens the airways (bronchodilator). It relaxes the muscles around the airways.

Quick-relief medications, also called rescue medications are used as needed for rapid, short-term relief of symptoms during an asthma attack, or before exercise. Types of quick-relief medications include:
Short-acting beta-2 agonists, such as albuterol. These inhaled medications, called bronchodilators, ease breathing by temporarily relaxing airway muscles. They act within minutes, and effects last four to six hours. Ipratropium (Atrovent). Like other bronchodilators, ipratropium relaxes the airways, making it easier to breathe. Ipratropium is mostly used for emphysema and chronic bronchitis.
Oral and intravenous corticosteroids to treat acute asthma attacks or very severe asthma. Examples include prednisone and methylprednisolone.

Medications for allergy-induced asthma.

These decrease the sensitivity to a particular allergen or prevent the immune system from reacting to allergens. Allergy treatments for asthma include:
Immunotherapy. Allergy-desensitization shots (immunotherapy) gradually reduce your immune system reaction to specific allergens.
Anti-IgE monoclonal antibodies, such as omalizumab (Xolair) reduces the immune system's reaction to allergens.

This invention will be better understood by reference to the Experimental Details that follow, but those skilled in the art will readily appreciate that these are only illustrative of the invention as described more fully in the claims that follow thereafter. Additionally, throughout this application, various publications are cited. The disclosure of these publications is hereby incorporated by reference into this application to describe more fully the state of the art to which this invention pertains.

### EXAMPLES

The following examples illustrate the invention. Other embodiments will occur to the person skilled in the art in light of these examples.

Inhibition of Asthmatic Airway Inflammation in Mice by M0014

Experimental Methods

BALB/c mice (n=6-8 per group) were sensitized to OVA by i.p. injection of OVA/alum (10 μg OVA grade V adsorbed to 1 mg aluminium hydroxide; Sigma-Aldrich) on days 0 and 7 and were subjected to OVA aerosol challenges (grade III) on days 17-19; aerosol challenges were dispensed from a jet nebulizer delivering 1% OVA in PBS for 30 minutes. Thirty minutes before each OVA exposure, mice were anesthetized using Avertin (Sigma-Aldrich) and received an i.t. injection of control vehicle, or of M0014 (0.1, 0.4 or 4 nM in PBS) in a volume of 80 μl. Twenty-four hours after the last OVA exposure, BAL was performed and LNs were resected and digested using collagenase/DNAse.

Flow cytometry and sorting. After counting and washing, BAL cells were stained for 30 minutes with FITC-labeled anti-I-Ad/I-Ed (macrophages/DCs), PE-labeled anti-CCR3 (eosinophils), Cy-chrome-labeled anti-CD3 and anti-CD19 (lymphocytes), and allophycocyanin-labeled (APClabeled) anti-CD11c (macrophages/DCs) in PBS containing 0.5% BSA and 0.01% sodium azide. Differential cell counts were analyzed by flow cytometry, as previously described (van Rijt et al., 2004).

Cytokine measurements. To measure cytokine levels, MLN cells were plated in round-bottomed 96-well plates (1×106 cells/ml) and restimulated with OVA (10 μg/ml) for 4 days. The presence of IL-4, IL-5, IL-13 and IFN-γ was assayed on supernatants by ELISA (BD).

For the measurement of dynamic resistance and compliance, mice were anesthetized with urethane, paralyzed using d-tubocurarine, tracheotomized, and intubated with an 18-gauge catheter, followed by mechanical ventilation with a Flexivent apparatus (SCIREQ). Respiratory frequency was set at 120 breaths per min with a tidal volume of 0.2 ml and a positive end-expiratory pressure of 2 ml H2O. Increasing concentrations of metacholine were administered via the jugular vein. Dynamic resistance and compliance was recorded after a standardized inhalation maneuver given every 10 seconds for 2 minutes. Baseline resistance was restored before administering the subsequent doses of metacholine.

Results

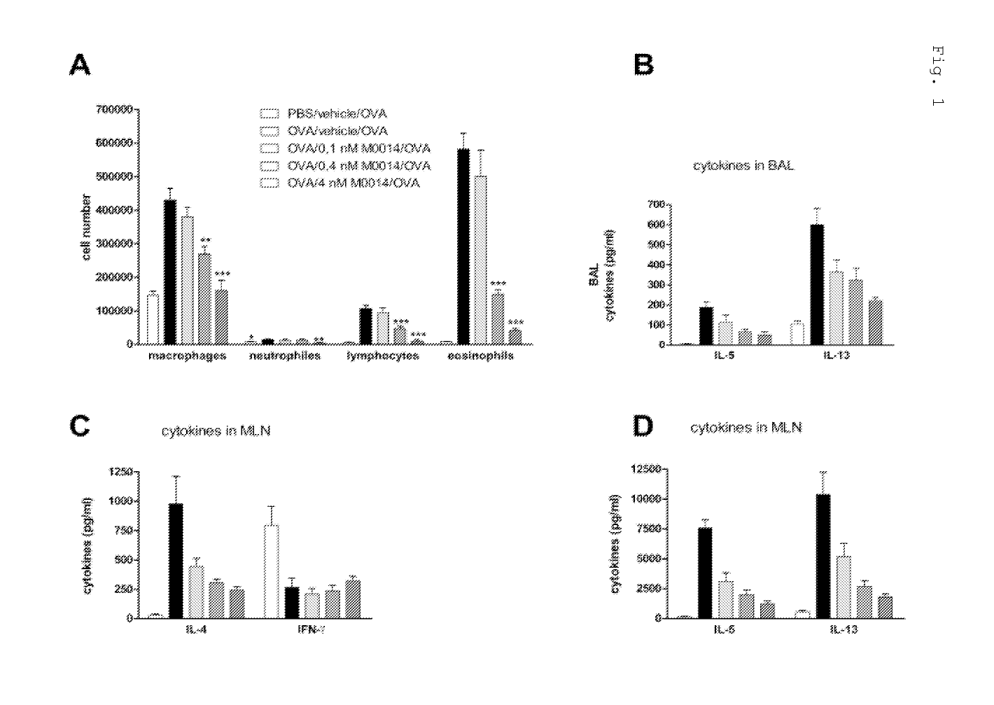

It was investigated whether local application of M0014 could influence the development of experimental asthma in already sensitized mice. Sensitization to OVA was induced using i.p. injection of OVA (or sham PBS) in the Th2 adjuvant alum, and mice were subsequently challenged 3 times 10 days later. As expected, OVA-sensitized mice treated with vehicle prior to OVA aerosol challenge (OVA/vehicle/OVA) developed bronchoalveolar lavage (BAL) fluid eosinophilia and lymphocytosis accompanied by enhanced Th2 cytokine production in the mediastinal LNs (MLNs), an effect not seen in sham-sensitized mice (PBS/vehicle/OVA; FIG. 1A). The intratracheal (i.t.) administration of M0014 (80 μl) 30 minutes prior to each allergen challenge resulted in a significant dose-dependent reduction of the macrophage, lymphocyte and eosinophil infiltrate into the BAL compartment (FIG. 1A). The reduction of airway inflammation in M0014-treated mice was accompanied by mildly but significantly reduced levels of IL-4, IL-5, and IL-13 in the MLNs and a weak increase in IFN-γ production (FIG. 1C and D).

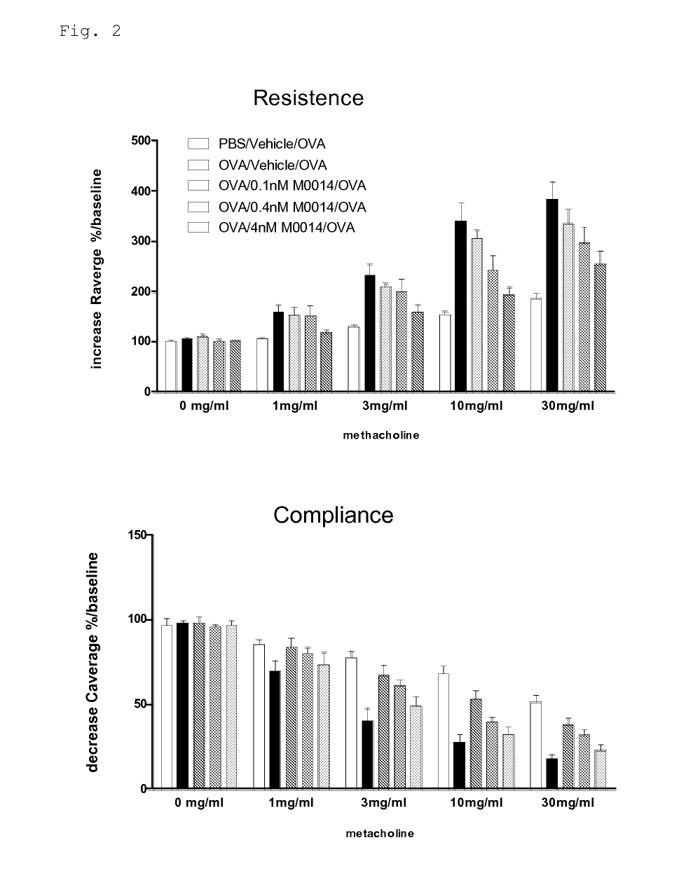

BHR to non-specific stimuli like metacholine is one of the defining symptoms of allergic asthma. As shown in FIG. 2, the allergen challenge of OVA-sensitized mice induced a significant change in responsiveness to i.v. metacholine compared with sham-sensitized mice, as measured 24 hours after the last OVA aerosol challenge by invasive measurement of dynamic resistance and compliance in mechanically ventilated mice. Inhalation of M0014 prior to each allergen challenge markedly attenuated the OVA-induced change in metacholine responsiveness.

Suppression of Inflammatory Cell Recruitment to the Lung by M0014

The below summarizes the results of two independent studies, of the oral administration of M0014 on zymosan induced inflammatory cell recruitment to the lung in an mouse model.

Experimental Methods—Neutrophil Recruitment to the Lung

Female Balb/c mice (7-8 weeks; 20 g) were used in all studies. The animals were kept in standard animal holding facilities and have unlimited access to food and water. Animals were randomized to receive vehicle or 0.1-0.2 ml/20 g mouse, M0014 (0.001, 0.01, 0.1 and 1 mg/kg) via the oral route 30 min prior to, and 6 h after the administration of zymosan (i.n; 20 μL to each nostril, to give a total volume of administration of 40 μL). Animals received zymosan A to give a total dose of 4 mg/mouse.

Drug Preparation

5 mg of M0014 was dissolved in 5 mL of sterile water to give 1 mg/ml solution. The solutions were prepared freshly each time. The 1 mg/ml solution was diluted to a 0.1, 0.01, 0.001 and 0.0001 mg/ml solution. From these concentrations, 0.2 ml was administered orally to obtain 1, 0.1, 0.01 and 0.001 mg/kg respectively. Sterile water used as control vehicle.

Experimental Protocol

The experimental design of the study was as follows:

Twenty-four hours after dosing, the mice were killed by overdose with an injectable anaesthetic and 0.5 ml of saline was injected into lung via a tracheal cannula and the fluid collected. This was repeated 3 times. Approximately 1 ml of lavage fluid was collected and stored on ice. Total cells and differential cells were counted and a reduction in neutrophil numbers was the primary end point.

Results

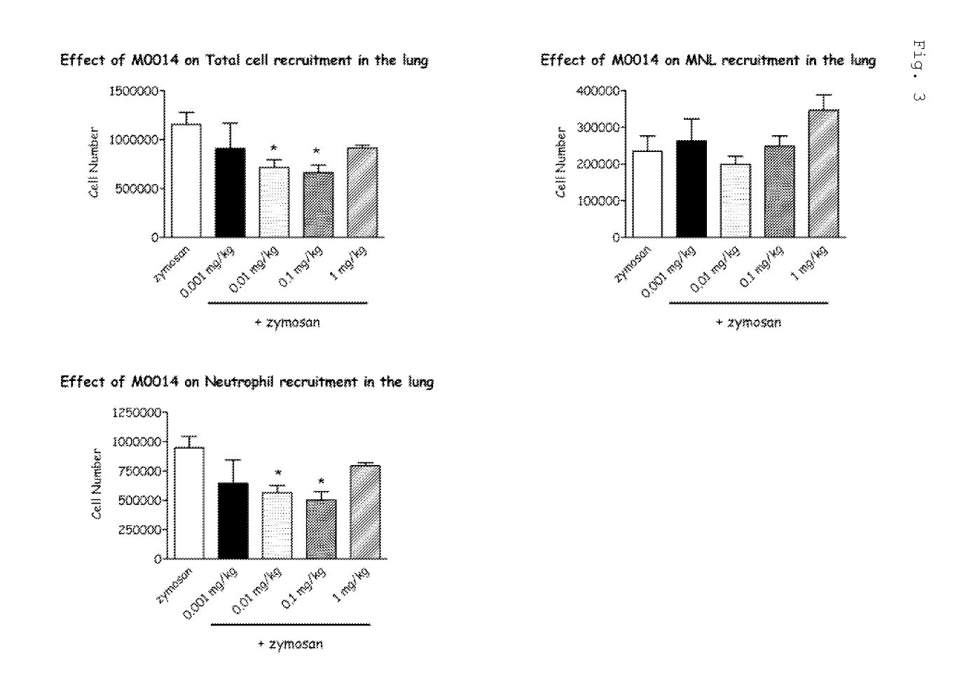

The total number of cells recruited to the airways was significantly reduced by M0014 (0.01 and 0.1 mg/kg; P<0.05, Dunnett's test versus control; FIG. 3a). This corresponded to a significant decrease in the recruitment of neutrophils to the airways by approximately 40-50% at 0.01 and 0.1 mg/kg (P<0.01; Dunnett's test vs control, FIG. 3c).

Discussion

As already mentioned hereinbefore, up till now, no direct effects have been observed for 5-HT4 R antagonists in for example, airway diseases like asthma. In animal models, the only 5-HT receptor claimed to be involved in the development of airway hyperresponsiveness (AHR) has been the 5-HT2A receptor (De Bie J J, Henricks P A, Cruikshank W W et al. Modulation of airway hyperresponsiveness and eosinophilia by selective histamine and 5-HT receptor antagonists in a mouse model of allergic asthma. Br J Pharmacol 1998; 124:857-64.1996; 304:15-21).

It has now been found, and different from earlier studies, that the 5-HT4 R is directly involved in the development of airway hyperresponsiveness (see FIG. 3), in that 5-HT4 R specific antagonists are capable to prevent and revert Bronchial Hyperreactivity in an animal model of asthmatic airway inflammation. In addition, antagonism of the 5HT4 receptor per se also reduced recruitment of neutrophils to the site of inflammation in a model of non-allergic inflammation.

These results have been confirmed in a recent publication of Segura, P. et al., that identify a direct involvement of the serotonin receptors 5-HT2A, 5-HT4 and 5-HT7 in the antigen induced airway hyperresponsiveness in guinea-pigs (P. Segura et al., Clin. & Exp. Allergy (2009) Dec. 3; 1-12). In this study a variety of 5-HT4 receptor antagonists and in particular GR113808 were capable to normalize airway hyperresponsiveness in this guinea pig model.

1. A 5-HT4 R antagonist for use in the treatment and/or prevention of airway diseases; in particular for use in the treatment and/or prevention of asthmatic airway inflammation and COPD.

2. The 5-HT4 R antagonist as claimed in claim 1, wherein said 5-HT4 R antagonist is selected from the group consisting of;

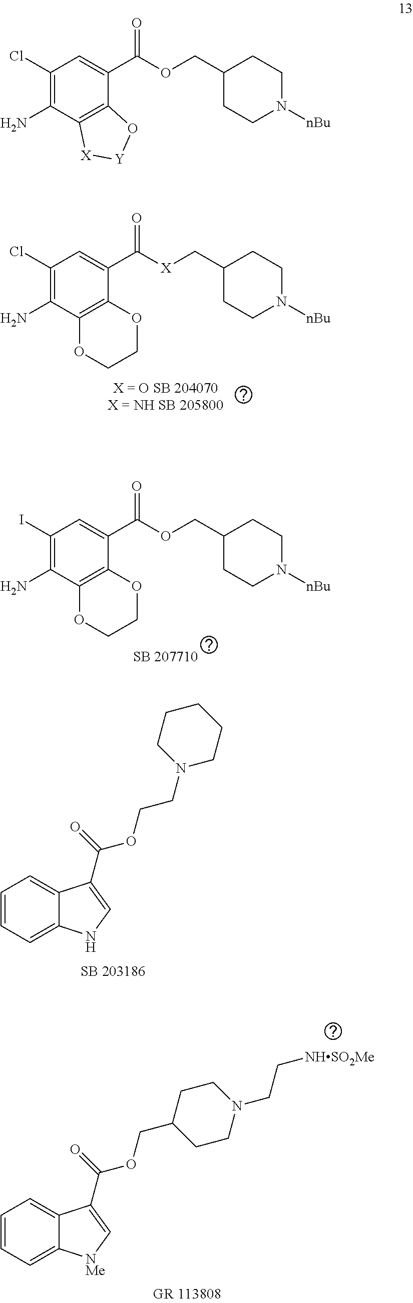

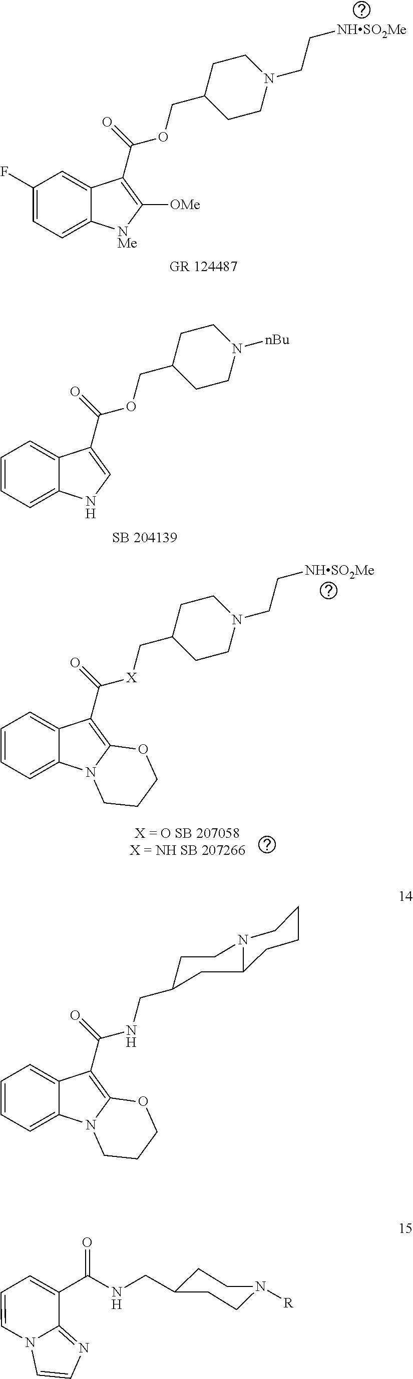

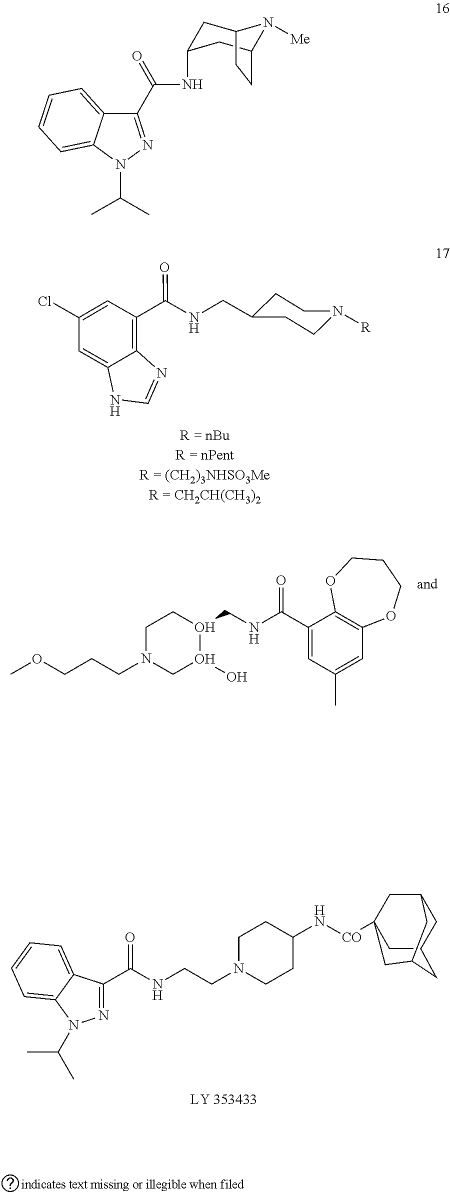

3. A compound of formula (I) 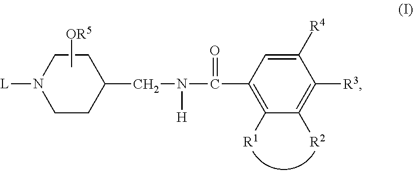
a stereochemically isomeric form thereof, an N-oxide form thereof, or a pharmaceutically acceptable acid or base addition salt thereof, wherein —R¹-R²-is a bivalent radical of formula
—O—CH₂—O— (a-1),
—O—CH2-CH2- (a-2),
—O—CH2-CH2-O— (a-3),
—O—CH2-CH2-CH2- (a-4),
—O—CH2-CH2-CH2-O— (a-5),
—O—CH2-CH2-CH2-CH2- (a-6),
—O—CH2-CH2-CH2-CH2-O— (a-7),
—O—CH2-CH2-CH2-CH2-CH2- (a-8),
wherein in said bivalent radicals optionally one or two hydrogen atoms on the same or a different carbon atom may be replaced by C₁₋₆alkyl or hydroxy,
R³ is hydrogen, halo, C₁₋₆alkyl or C₁₋₆alkyloxy;
R⁴ is hydrogen, halo, C₁₋₆alkyl; C₁₋₆alkyl substituted with cyano, or C₁₋₆alkyloxy; C₁₋₆alkyloxy; cyano; amino or mono or di(C₁₋₆alkyl)amino;
R⁵ is hydrogen or C₁₋₆alkyl and the —OR⁵ radical is situated at the 3- or 4-position of the piperidine moiety; L is hydrogen, or L is a radical of formula
-Alk-R⁶ (b-1),
-Alk-X—R⁷ (b-2),
-Alk-Y—C(═O)—R⁹ (b-3),
-Alk-Z—C(═O)—NR¹¹R¹² (b-4)
-Alk-C(═O)—NH—C(═O)—R¹³ (b-5),
-Alk-C(═O)—NH—SO₂—R¹³ (b-6),
-Alk-SO₂—NH—C(═O)—R¹³ (b-7),
-Alk-SO₂—NH—SO₂—R¹³ (b-8),
wherein each Alk is C₁₋₁₂alkanediyl; and
R⁶ is hydrogen; hydroxy; cyano; C₃₋₆cycloalkyl; C₁₋₆alkylsulfonylamino; aryl; aminosulfonyl optionally substituted with C₁₋₄alkyl, C₃₋₆cycloalkyl or phenyl; or Het;
R⁷ is C₁₋₆alkyl; C₁₋₆alkylsulfonyl; C₁₋₆alkyl substituted with hydroxy; C₃₋₆cycloalkyl; aryl or Het;
R⁹ is hydrogen, C₁₋₆alkyl, C₁₋₆alkylsulfonylamino, C₃₋₆cycloalkyl, hydroxy or aryl;
X is O, S, SO₂ or NR⁸; said R⁸ being hydrogen or C₁₋₆alkyl; R⁹ is hydrogen, C₁₋₆alkyl, C₁₋₆alkylsulfonylamino, C₃₋₆cycloalkyl, hydroxy or aryl;
Y is a direct bond, O, S, or NR¹⁰ wherein R¹⁰ is hydrogen or C₁₋₆alkyl;
Z is a direct bond, O, S, or NR¹⁰ wherein R¹⁰ is hydrogen or C₁₋₆alkyl;
R¹¹ and R¹² each independently are hydrogen, C₁₋₆alkyl, C₃₋₆cycloalkyl, or R¹¹ and R¹² combined with the nitrogen atom bearing R¹¹ and R¹² may form a pyrrolidinyl, piperidinyl, piperazinyl or 4-morpholinyl ring both being optionally substituted with C₁₋₆alkyl;
R¹³ is C₁₋₆alkyl or phenyl;
aryl represents unsubstituted phenyl or phenyl substituted with 1, 2 or 3 substituents each independently selected from halo, hydroxy, C₁₋₆alkyl, C₁₋₆alkyloxy, C₁₋₆alkylcarbonyl, nitro, trifluoromethyl, amino, aminocarbonyl, hydroxycarbonyl, and aminosulfonyl; and
Het is furanyl; furanyl substituted with C₁₋₆alkyl or halo; tetrahydrofuranyl; tetrahydrofuranyl substituted with C₁₋₆alkyl; dioxolanyl; dioxolanyl substituted with C₁₋₆alkyl; dioxanyl; dioxanyl substituted with C₁₋₆alkyl; tetrahydropyranyl; tetrahydropyranyl substituted with C₁₋₆alkyl; 2,3-dihydro-2-oxo-1H-imidazolyl; 2,3-dihydro-2-oxo-1H-imidazolyl substituted with one or two substituents each independently selected from halo, or C₁₋₆alkyl; pyrrolidinyl; pyrrolidinyl substituted with one or two substituents each independently selected from halo, hydroxy, or C₁₋₆alkyl; pyridinyl; pyridinyl substituted with one or two substituents each independently selected from halo, hydroxy, C₁₋₆alkyl; pyrimidinyl; pyrimidinyl substituted with one or two substituents each independently selected from halo, hydroxy, or C₁₋₆alkyl; pyridazinyl; pyridazinyl substituted with one or two substituents each independently selected from hydroxy, C₁₋₆alkyloxy, C₁₋₆alkyl or halo; pyrazinyl; pyrazinyl substituted with one ore two substituents each independently selected from hydroxy, C₁₋₆alkyloxy, C₁₋₆alkyl or halo.; morpholinyl; morpholinyl substituted with C₁₋₆alkyl; tetrazolyl; tetrazolyl substituted with halo, hydroxy, or C₁₋₆alkyl; pyrazolyl; pyrazolyl substituted with halo, hydroxy, or C₁₋₆alkyl; isoxazolyl; isoxazolyl substituted with halo, hydroxy, or C₁₋₆alkyl; isothiazolyl; isothiazolyl substituted with halo, hydroxy, or C₁₋₆alkyl; 2,4-dioxo-imidazolidinyl; 2,4-dioxo-imidazolidinyl substituted with one or two substituents each independently selected from halo, or C₁₋₆alkyl; oxazolyl; oxazolyl substituted with halo, hydroxy, or C₁₋₆alkyl; thiazolyl; thiazolyl substituted with halo, hydroxy, or C₁₋₆alkyl; or pyranyl; pyranyl substituted with halo, hydroxy, or C₁₋₆alkyl; for use in the treatment and/or prevention of airway diseases; in particular for use in the treatment and/or prevention of asthmatic airway inflammation.

4. A compound according to claim 3 wherein;
the —OR⁵ radical is situated at the 3- or 4-position of the piperidine moiety; the absolute configuration of the piperidine moiety is (3S,4S);
L is a radical of formula (b-1), (b-2), (b-6) or (b-8); more in particular L is a radical of formula (b-2);
Alk is C₁₋₄alkanediyl; 1,3-propanediyl or 1,4-butanediyl; more in particular Alk is C₁₋₄alkanediyl;
—R¹-R²-is a bivalent radical of formula (a-5);
R³ is hydrogen, halo, or C₁₋₄alkyl; more in particular R³ is hydrogen;
R⁴ is halo or C₁₋₆alkyl; more in particular R⁴ is C₁₋₆alkyl;
R⁵ is hydrogen or C₁₋₆alkyl; more in particular R⁵ is hydrogen and the —OR⁵ radical is situated at the 3-position of the piperidine moiety having the trans configuration;
R⁶ is Het, aminosulfonyl, or aminosulfonyl substituted with C₁₋₄alkyl or phenyl; more in particular R⁶ is Het;
R⁷ is aryl or C₁₋₆alkyl;
R¹³ is C₁₋₄alkyl; and
Het is morpholinyl; pyrazolyl substituted with hydroxy; isoxazolyl substituted with hydroxy; 2,4-dioxo-imidazolidinyl; tetrazolyl; or tetrazolyl substituted with hydroxy;
for use in the treatment and/or prevention of airway diseases; in particular for use in the treatment and/or prevention of asthmatic airway inflammation.

5. A compound according to claim 3 wherein;
—R¹-R²— is a radical of formula (a-5); R³ is hydrogen; R⁴ is methyl; R⁵ is hydrogen; and
L is a radical of formula (b-2), wherein X is O, Alk is C₁₋₄alkanediyl and R⁷ is C₁₋₆alkyl;
for use in the treatment and/or prevention of asthmatic airway inflammation.

6. (3S-trans)-8-methyl-3,4-dihydro-3H-benzo[b][1,4]dioxepine-6-carboxylic acid [3-hydroxy-1-(3-methoxy-propyl)-piperidine-4-ylmethyl]-amide; for use in the treatment and/or prevention of airway diseases; in particular for use in the treatment and/or prevention of asthmatic airway inflammation and COPD.
